(19) United States
(12) Patent Application Publication
(10) Pub. No.: US 2015/0238525 A1
(43) Pub. Date: Aug. 27, 2015

(54) INJECTABLE STERILE AQUEOUS FORMULATION BASED ON CROSSLINKED HYALURONIC ACID AND ON HYDROXYAPATITE, FOR THERAPEUTIC USE

(71) Applicant: APTISSEN S.A., Plan-les-Ouates (CH)

(72) Inventor: Samuel Gavard Molliard, Bogeve (FR)

(73) Assignee: APTISSEN S.A., Plan-les-Ouates (CH)

(21) Appl. No.: 14/433,089

(22) PCT Filed: Sep. 24, 2013
(86) PCT No.: PCT/EP2013/069877

§ 371 Date: Apr. 2, 2015

(30) Foreign Application Priority Data
Oct. 8, 2012 (FR) 1259582

(51) Int. Cl. A61K 33/42; A61K 9/06; A61K 31/167; A61K 9/00; A61K 31/738; A61K 45/06

(52) U.S. Cl. CPC A61K 33/42; A61K 31/738; A61K 45/06; A61K 31/167; A61K 9/0024; A61K 9/06

(57) ABSTRACT

This invention relates to an absorbable sterile injectable aqueous formulation that is ready to use, used for therapeutic purposes as a cohesive particle-based viscoelastic gel containing i) cross-linked hyaluronic acid, or one of its salts, at a concentration between 1% and 4% (mass/volume), with the cross-linking that is performed making it possible to obtain a gel having a base of cross-linked hyaluronic acid having a so-called cohesive structure, and ii) hydroxyapatite. at a concentration between 10% and 70% (mass/volume), said hydroxyapatite being in the form of particles having an average size less than or equal to 650 μm; with the said sterile injectable aqueous formulation having viscoelastic properties such that Tan δ at a frequency of 1 Hz is less than or equal to 0.60.

### DOMAIN OF THE INVENTION

This invention relates to an absorbable sterile injectable aqueous formulation, ready to use, used for therapeutic purposes as a cohesive particle-based viscoelastic gel comprising i) cross-linked hyaluronic acid or one of its salts at a concentration between 1% and 4% (mass/volume); the cross-linking that is performed makes it possible to obtain a cross-linked hyaluronic acid gel having the said cohesive structure, and ii) hydroxyapatite at a concentration between 10% and 70% (mass/volume), said hydroxyapatite being in the form of particles having an average size less than or equal to 650 μm; said sterile injectable aqueous formulation having viscoelastic properties such that Tan δ at a frequency of 1 Hz is less than or equal to 0.60.

### CONTEXT OF THE INVENTION

This invention relates to the field of surgery in humans or animals and in particular to orthopedic surgery, dental or maxillofacial surgery, ENT (Ear, Nose, and Throat) surgery, urological or gynecological surgery, and gastroenterological surgery.

Many biomaterials have been developed for surgery to fill in and/or to restore volume, and/or to replace biological tissue for a long- or short-term period.

Within these biomaterials, there are nonabsorbable solutions such as hip prostheses made of metallic material used in orthopedic surgery or absorbable solutions such as, for example, the bioresorbable polymer stents used in cardiac surgery.

Targeted biomaterial fillers can be used for a variety of therapeutic applications in soft or hard tissue. For example, one may cite:
Maxillofacial surgery: reconstruction of the orbital cavity after enucleation using a ball made of ceramic material;
Dental surgery: treatment of parodontitis through the application of ceramic materials to fill periodontal pockets;
Urological surgery: treatment of urinary incontinence or vesicoureteral reflux by endo-urethral injection of fillers such as collagen-based products;
Gastroenterological surgery: treatment of fecal incontinence by injecting filler products such as silicone-based products;
ENT surgery: treatment of the vocal cords in order to improve phonation, through the injection of filler products such as, for example, carbomer-based products;
Orthopedic surgery: treatment of bone defects through the use of a porous ceramic-based bone substitute;

In general, it can be said that implanted targeted filler products can treat the desired tissues:
in the case of soft tissues, by mainly creating a long-term filling/increase in these tissues.
in the case of hard tissues, by mainly favoring the construction/regeneration of these tissues.

Among the so-called resorbable targeted filler biomaterials, products containing hydroxyapatite particles may be mentioned as an example.

Hydroxyapatite has a chemical composition which is very similar to that of the mineral phase of bone. Its biological properties and its biocompatibility make it an excellent bone-substitute product. Bone colonization by the substitute is highly dependent upon the porous characteristics of the material and in particular on pore size and distribution, and the interconnection between macropores (number and size). The interconnections are tunnels that allow the passage of cells and the circulation of blood between the pores and thus promote bone formation within the substitute.

Hydroxyapatite, due to its high biocompatibility and its slow absorption within the body, is administered into very different tissues and for various applications under different forms such as:
Hydroxyapatite-based cements. These cements are prepared by the surgeon in the operating room (malleable material during the few minutes' preparation) and then administered to the treatment area (the material hardens in situ). They are used as bone substitutes due to their good osseointegration and their bioresorbability, which leaves room for bone neoformation over time.
Hydroxyapatite-based solutions or gels that may contain a polymer such as carboxymethylcellulose. These products can be used in various fields such as to fill periodontal defects in dental surgery or to treat urinary incontinence, or even as a filler in orthopedic surgery after curettage of cysts or benign tumors.
Hydroxyapatite-based products are available in the block, powder, and granular form used in orthopedics and possessing rapid osseointegration, used for example as a filler in hip replacement or, in maxillofacial repair, as a supplement to dental implants.

Among the hydroxyapatite-based products, there are many that are not ready to use and that require advance preparation by the surgeon (in the case of hydroxyapatite cements which requires pre-mixing of a powder and a solution within a specified time before placing it on or in the area to be treated, and the curing of the cement in situ). Other products need to be prepared by the surgeon (solid material to be “cut” to the proper shape) before implantation on or in the treatment area. Issues related to the migration of hydroxyapatite particles have also been reported. These problems can generate complications and/or loss of performance of the product. For example, the migration of hydroxyapatite particles induces a loss of the filler effect in biological tissues and can potentially cause side effects. Indeed, hydroxyapatite particles can be chiefly concentrated in certain areas more or less removed from the treatment area due to the mechanical stresses to which the biomaterial is subjected. The presence of particles or a too-high particle concentration in an unwanted area can lead to more or less severe complications.

Hyaluronic acid (HA) is another resorbable biomaterial found in many products, in particular those used as fillers. It is used in its native form (not chemically modified) or in cross-linked form in many therapeutic and aesthetic areas.

Cross-linked hyaluronic acid is well known in dermo-aesthetic applications, a domain in which it is injected into or under the dermis to fill wrinkles or to restore volume in different areas of the body for a period of several months. It has the advantage of having very few post-injection side effects and extremely rare complications in the long term. On the other hand, in case of a poorly applied injection, the practitioner has the opportunity to correct his treatment by injecting a solution of hyaluronidase (HA-specific enzymes). This solution will degrade the previously injected cross-linked HA-based product. Due to the gradual disappearance of cross-linked HA injections (resorption of the polymer into the tissues over time); they must be repeated at regular intervals, typically every 6 to 12 months, in order to maintain the effectiveness of the treatment. However, non-cross-linked hyaluronic acid has a short residence time in the skin (with a half-life of less than one week); it is degraded in vivo by various factors such as radical, enzymatic, thermal, and mechanical degradation. It is the cross-linking that can significantly increase its half-life by slowing the kinetics of hyaluronic acid degradation by the factors described above, thus allowing an effective aesthetic treatment lasting as long as about 12 months.

Cross-linked or non-cross-linked hyaluronic acid is used in therapeutic areas such as:
Orthopedics, where it is administered as viscosupplementation in joints affected by osteoarthritis, to reduce pain and increase the mobility of the treated patient,
In ophthalmology, as a viscoelastic solution during cataract surgery to create an intraocular space and to protect the tissues of the eye, or as a drainage implant in the area of glaucoma surgery,
In urology, as a filler to treat urinary or fecal incontinence, or
In maxillofacial surgery, to reconstruct the orbital cavity after enucleation.

Intensive scientific research is being conducted worldwide to develop hyaluronic acid-based treatments with enhanced performance over time. The goal is to have products capable of degrading less rapidly in biological tissues in order to maintain an optimal filler effect over the longest period possible, while maintaining a high level of safety of the injected products. Moreover, hyaluronic acid has many advantages, making it a biomaterial of choice for various medical applications. However, it unfortunately does not possess properties that impart to it a strong effect in the field of osteosynthesis for bone reconstruction, unlike a biomaterial such as hydroxyapatite.

In this context, it is important to provide practitioners with ready-to-use biocompatible formulations that are easy to administer and that possess outstanding mechanical properties and provide a long-term effect suitable for injection for therapeutic purposes without causing complications related to migration of the implanted product.

### ABSTRACT OF THE INVENTION

The invention relates to a sterile injectable and bioresorbable aqueous formulation, ready to use, for therapeutic purposes, in the form of a cohesive particle-based viscoelastic gel containing i) cross-linked hyaluronic acid or one of its salts at a concentration between 1% and 4% (mass/volume); the cross-linking that is performed makes it possible to obtain a gel containing cross-linked hyaluronic acid having the said cohesive structure, and ii) hydroxyapatite at a concentration between 10% and 70% (mass/volume), said hydroxyapatite being in the form of particles having an average size less than or equal to 650 μm; said sterile injectable aqueous formulation having viscoelastic properties such that Tan δ at a frequency of 1 Hz is less than or equal to 0.60.

In accordance with another goal, the present invention concerns a process for the preparation of a sterile injectable aqueous formulation comprising the following steps: a) preparation of a first mixture comprising at least 1% to 4% by weight of cross-linked hyaluronic acid or a salt thereof, by the formation of covalent bonds between the chains of the said biopolymer using bi- or polyfunctional molecules, with the cross-linking that is performed making it possible to obtain a gel containing cross-linked hyaluronic acid having the said cohesive structure, b) purification of the said first mixture, c) the subsequent addition of hydroxyapatite at a concentration between 10% to 70% (mass/volume) by homogeneously dispersing it in the cross-linked hyaluronic acid-based gel, d) placing the gel thus obtained in a ready-to-use form, e) sterilization of the product with moist heat.

Also according to another goal, this invention relates to a kit preferably supplied in the form of a syringe containing the formulation as described above.

### BRIEF DESCRIPTION OF THE FIGURE

FIG. 1 shows pictures of the comparison of the gels B′, X, and of the CMC-based formulation and of the hydroxyapatite, in accordance with the test described in Example 2.

### DETAILED DESCRIPTION OF THE INVENTION

The invention described below serves the purpose of providing a new ready-to-use, sterile injectable aqueous bioabsorbable formulation, used for therapeutic purposes and with the specific properties of viscoelasticity, filling, and long-term performance, and, in certain cases, an ability to actively promote the restoration of the surrounding biological tissues. This formulation is characterized in that it is in the form of a cohesive particle-based viscoelastic gel containing:

i) cross-linked hyaluronic acid or one of its salts at a concentration between 1% and 4% (mass/volume); the cross-linking that is performed makes it possible to obtain a gel containing cross-linked hyaluronic acid having the said cohesive structure, and

ii) hydroxyapatite at a concentration between 10% and 70% (mass/volume), said hydroxyapatite being in the form of particles having an average size less than or equal to 650 μm;

with the said sterile injectable aqueous formulation having viscoelastic properties such that Tan δ at a frequency of 1 Hz is less than or equal to 0.60.

The new formulation of this invention is bioresorbable. It includes biomaterials that are more or less long-term biocompatible and assimilable by biological tissues. In many applications, particularly in the field of bone reconstruction in orthopedics, it can play the role of promoting the regrowth of the surrounding biological tissue. In the case of bone-reconstruction applications, the formulation according to the invention is resorbed, thus leaving room for a neoformed bone over time.

In an altogether surprising manner, it was found that this formulation has a remarkable capacity for filling and/or the restoration of volume and/or the replacement of biological tissues over the long term, due to the synergy between the cross-linked hyaluronic acid and the hydroxyapatite particles, under the conditions of the invention.

From a mechanical point of view, the hydroxyapatite particles (solid performance: substantial elasticity and negligible viscosity) greatly enhance the elasticity of the gel and therefore its ability to create volume by inducing a significant force/pressure on the tissues in order to correct the deficient area to be treated.

The cross-linked hyaluronic acid provides viscoelastic properties, i.e., elasticity, but also viscosity, allowing it to have a gel consistency approximating that of the soft tissues and thus counterbalancing the very high degree of elasticity and the absence of viscosity provided by the hydroxyapatite particles. This makes it possible to have a product that integrates into the tissues in a much more homogeneous manner that is less traumatic for the tissues (strong limitation of post-injection inflammation), and that is less painful upon injection.

Furthermore, the cross-linked hyaluronic acid, under the conditions of the invention, will make it possible to reduce considerably the migration of the hydroxyapatite particles, which are held in the gel due to the strong cohesiveness provided by the cohesive cross-linked hyaluronic acid (cross-linked hyaluronic acid has low resorption kinetics). This strong limitation on migration makes it possible to have:
An improved long-term gel with an volumizing capacity,
A reduction in side effects by allowing the hydroxyapatite particles to remain at the injection site.

Hyaluronic acid is a polysaccharide made up of repeating disaccharide units of glucuronate and N-acetyl glucosamine. It is distributed widely among connective, epithelial, and neural tissues in humans as well as in animals. It is one of the main components of the extracellular matrix. It contributes significantly to the proliferation and migration of cells. It is chiefly found at high concentrations within the aqueous humor, synovial fluid, the skin, and the umbilical cord.

The preferred hyaluronic acid salts according to the invention include hyaluronic acid salts with a cation: for example, a mono- or divalent salt such as sodium, potassium, magnesium, calcium, or manganese salts. Sodium salts are especially preferred.

According to the invention, hyaluronic acid or a salt thereof is in cross-linked form. This cross-linking is obtained through the formation of covalent bonds between the chains of the said biopolymer, with the aid of bifunctional or polyfunctional molecules; the cross-linking that is performed makes it possible to obtain a cross-linked hyaluronic acid-based gel that has a so-called cohesive structure, also known as a monophasic structure.

In a non-limitative example of the present invention, the cross-linked hyaluronic acid is made from fibers of sodium hyaluronate placed in contact with butanediol diglycidyl ether (BDDE) to form a gel network.

The cohesive nature of the cross-linked hyaluronic acid-based gel is an important and necessary specificity of the invention. The gel should not disintegrate rapidly when introduced into water, as a gel having a non-cohesive nature does, also called a biphasic gel (a cross-linked hyaluronic acid-based gel that cannot hold the hydroxyapatite particles and thus prevent migration). Example 2 points out this difference between a cohesive gel and a non-cohesive gel.

This invention generally comprises a concentration of cross-linked hyaluronic acid, or of a salt thereof, from 1% to 4% (mass/volume), preferably between 1% and 3% (mass/volume). According to an especially preferred variant, the concentration of the cross-linked hyaluronic acid or a salt thereof is between 1.5% and 2.5% (mass/volume). Alternatively, the concentration of cross-linked hyaluronic acid, or of a salt thereof, may be between 1.5% and 3% (mass/volume), or between 1% and 2.5% (mass/volume).

Advantageously, the aqueous formulation according to the invention includes hyaluronic acid or a salt thereof, whose molecular weight is preferably between 2.5×10⁵ Da and 4×10⁶ Da. According to an especially preferred variant, this molecular weight is between 1×10⁶ Da and 3×10⁶. Alternatively, the molecular weight is between 1×10⁶ Da and 2.5×10⁶, or between 2.5×10⁵ Da and 3×10⁶ Da.

Hydroxyapatite is a mineral species of the phosphate family, having the formula Ca5(PO4)3(OH), usually written as Ca10(PO4)6(OH)2 to stress the fact that the lattice of the crystalline structure contains two molecules. Hydroxyapatite belongs to the crystallographic apatite family, which are isomorphic compounds having the same hexagonal structure. This compound has been used as a biomaterial for many years in various medical specialties.

This invention generally includes a concentration of hydroxyapatite particles that is between 10% and 70% (mass/volume), preferably between 20 and 60% (mass/volume), preferably between 30 to 50% (mass/volume), and the average hydroxyapatite particle size is less than or equal to 650 μm, preferably less than or equal to 200 μm, less than or equal to 80 μm, or less than or equal to 500 nm.

It has been observed that the viscosity and elasticity properties of the formulation according to the invention are optimal when the parameter Tan delta or Tan δ, corresponding to the ratio [modulus of viscosity G″/modulus of elasticity G′] at the frequency of 1 Hz is 0.60 or less, and preferably 0.58 or less. Indeed, it has been shown that the elastic nature of the formulation according to the invention, compared to its viscosity, must be great enough to prevent the sedimentation of the hydroxyapatite particles. Thus, it was observed that above 0.60, the hydroxyapatite particles tend to settle over time. This sedimentation involves obtaining a formulation having a base of non-homogeneous hydroxyapatite particles. This formulation is unsatisfactory, on the one hand, for the act of injecting the formulation through a needle (due to clogging of the needle), and, on the other hand, for the safety and performance of the formulation in the injection zone (for example, due to the creation of areas of hard tissue induced by the concentration of the hydroxyapatite particles in certain areas that are more or less far from the area to be treated, due to the mechanical stresses to which the biomaterial is subjected. The presence of particles or a too-high particle concentration in an unwanted area can lead to more or less severe complications).

In general, the measurement of the elasticity (G′) and the ratio between the viscosity and the elasticity (Tan delta=G″/G′) is taken by performing a frequency sweep from 0.01 to 100 Hz using a rheometer with a plate geometry of 40 mm, a working air gap of 1000 micrometers, and an analysis temperature of 25° C.

As shown in Example 2, the cohesiveness of the formulation according to the invention is a major element, but it is equally necessary that its viscoelastic nature be suitable in order to:
Avoid the sedimentation of the hydroxyapatite particles over time in its container, and
Avoid having a product that will separate into 2 phases (hydroxyapatite particles and the cross-linked hyaluronic acid gel) during injection and/or at the injection site, creating heterogeneities at the treated site.

Another goal of the invention is to provide better longevity over time in comparison with the formulations of the prior art. The improved longevity of the filler in the area to be treated is achieved through the ability of the cross-linked hyaluronic acid to hold the hydroxyapatite particles at the injection site over the long term, and through the ability of the hydroxyapatite particles to confer remarkable mechanical/rheological properties over the long term. The gain in clinical longevity is probably several months.

It is also important to note that the presence of the hydroxyapatite particles, which are radiopaque, imparts an advantage to the gel, because they can easily be located by the practitioner via radiography during and/or after the injection.

The opportunity for the practitioner to inject a solution of hyaluronidase to correct the injection and to degrade the cross-linked hyaluronic acid that makes up the product also imparts an advantage to the invention. This injection does not, however, accelerate the resorption of the hydroxyapatite particles; thus, there is no complete degradation of the product within the tissues.

Thus, this invention consists of a formulation as described above, used for filling and/or for the restoration of volume and/or replacement and/or regeneration of biological tissues, such as, for example:
In maxillofacial surgery to reconstruct the orbital cavity;
In dental surgery to treat parodontitis;
In urological surgery to the treat urinary incontinence or vesicoureteral reflux by endo-urethral injection;
In gastroenterological surgery to treat fecal incontinence;
In ENT surgery to treat the vocal cords to improve phonation;
In orthopedic surgery to treat bone defects.

The formulation according to the invention is generally used as is; however, the addition of at least one other additive (other than those mentioned above) and/or at least one active ingredient is not ruled out.

Thus, the formulation may further include one or more ceramic materials. These materials are generally selected from the group consisting of tricalcium phosphate, calcium carbonate, and calcium sulfate, or a combination of several of these ceramic materials.

The formulation according to the invention may further include one or more growth factors, such as those in the family of “bone morphogenetic proteins” (BMPs) and/or the family of “transforming growth factors b” (TGF-βs). In the large family of TGF β growth factors, the morphogenic growth factors (BMPs) have a specific effect on osteogenesis. BMPs can induce bone formation; they are osteoinductive biomaterials. They are present in infinitesimal quantities in the skeleton (1 μg/kg of bone). The development of molecular biology and, in particular, of cloning techniques has made it possible to produce these factors in unlimited and very pure quantities through genetic engineering in the rhBMP-2 recombinant form.

The formulation according to the invention may also include one or more anesthetics, selected from the group including lidocaine, either alone or in combination with adrenaline; procaine; etidocaine, either alone or in combination with adrenaline; articaine, either alone or in combination with adrenaline; mepivacaine; pramocaine; quinisocaine; or one or more of the salts of these anesthetics. According to an especially preferred variant, the anesthetic chosen is lidocaine hydrochloride. The presence of an anesthetic in the formulation according to the invention is of major interest to improve patient comfort during and after the injection.

According to another particular embodiment of the invention, the formulation according to the invention may also include one or more antioxidants, such as the antioxidants in the polyol family. The antioxidant may be chosen from the polyol group chiefly consisting of sorbitol, glycerol, mannitol, or propylene glycol.

According to another goal, the present invention concerns a process for the preparation of a sterile injectable aqueous formulation comprising the following steps: a) preparation of a first mixture containing at least 1% to 4% by weight of cross-linked hyaluronic acid or a salt thereof, through the formation of covalent bonds between the chains of said biopolymer with the aid of bi- or polyfunctional molecules, with the cross-linking that is performed making it possible to obtain a gel containing cross-linked hyaluronic acid having the said cohesive structure, b) purification of the said first mixture, c) the subsequent addition of hydroxyapatite at a concentration between 10% to 70% (mass/volume) by homogeneously releasing it into the cross-linked hyaluronic acid-based gel, d) placing the gel thus obtained into a ready-to-use form, e) sterilization of the product with moist heat.

Sterilization of the formulation according to stage e) is performed with moist heat. Those skilled in the art will select a heat sterilization cycle (temperature and duration of the sterilization cycle) suitable for sterilization of the product. For example, the following moist-heat sterilization cycles can be used: 131° C. for 1 minute; 130° C. for 3 minutes; 125° C. for 7 minutes; 121° C. for 20 minutes.

According to another goal, this invention relates to a kit preferably supplied in the form of a syringe containing the formulation as described above.

Advantageously, the formulation is ready to use; it can be injected directly by the practitioner. The type of injection depends on the site and/or the biological tissues concerned. The invention can be used in many therapeutic applications, including:
In maxillofacial surgery to reconstruct the orbital cavity;
In dental surgery to treat parodontitis;
In urological surgery to treat urinary incontinence or vesicoureteral reflux through endo-urethral injection;
In gastroenterological surgery to treat fecal incontinence;
In ENT surgery to treat vocal cords to improve phonation;
In orthopedic surgery to treat bone defects.

The present invention also relates to a kit in the form of a container other than a syringe, such as an ampoule or a vial containing the formulation as described above.

The invention will now be illustrated non-limitatively through the following examples 1 to 4:

### EXAMPLES

### Example 1

### Preparation of a Cross-Linked Hyaluronic Acid-Based Gel with a so-Called Cohesive Structure

Stage 1:

3.5 g of sodium hyaluronate with a molecular weight of 2.6 MDa was added to 1% sodium hydroxide (30.5 g). The mixture was allowed to homogenize for 1 hr 30 min. 420 mg butanediol diglycidyl ether (BDDE) was added to the homogenized mixture, sealed and placed in a water bath at 50° C. for 2 hours. The mixture was then neutralized by adding 7.5 g of 1N HCl.

The gel was purified by dialysis for 24 hours with an iso-osmolar saline solution that had a neutral pH (regenerated cellulose, separation limit: molecular weight=60 kDa) to obtain a hyaluronic acid concentration of 25 mg/ml (2.5%). It was then homogenized in a conventional paddle mixer for 1 hr 30 min (=gel A1/124 g).

The gel could then be degassed, packed into 2 ml glass syringes, and sterilized by steam autoclaving at 130° C. for 3 minutes (=gel A/viscoelastic gel having a so-called cohesive or monophasic structure).

Stage 2:

Preparation of the gel according to the invention. In 100 g of gel A1, 42.9 g of phosphocalcium hydroxyapatite Ca10(PO4)6(OH)2 was added, the particles of which had an average size of between 30 and 50 microns. The gel was then homogenized in a conventional paddle mixer for 1 hr 30 min (=gel B1/142.9 g).

The gel could then be degassed, packed into 2 ml glass syringes, and sterilized by steam autoclaving at 130° C. for 3 minutes (=gel B).

The gel was a cohesive particle-based viscoelastic gel. Indeed, it was in the form of a viscoelastic gel (it had elastic properties G′ and viscosity properties G″; see below), possessing strong cohesiveness (see Example 2) and containing hydroxyapatite particles.

The concentration of hyaluronic acid in the gel was 17.5 mg/ml (1.75%) (carbazole assay, European Pharmacopoeia method). Furthermore, the pH (7.15) and osmolarity (315 mOsm/kg) of the gel were physiological.

The gel was easily injectable through a needle. A force of 26.3 N was required to push the gel through a 21 G needle, assuming a push speed of 12.5 mm/minute.

Gels A and B were characterized from a mechanical/rheological viewpoint. The rheometer used to perform these characterizations was an AR2000 (TA Instruments) with a plate geometry of 40 mm, a working air gap of 1000 micrometers, and an analysis temperature of 25° C.

A measurement of the elasticity (G′) and of the ratio between viscosity and elasticity (Tan delta=G″/G′) was taken by performing a frequency sweep from 0.01 to 100 Hz. A comparison of the parameters was performed at 1 Hz.

| Gel | G′ (1 Hz), in pA | Tan delta (1 Hz) |
|---|---|---|
| A | 184 | 0.25 |
| B | 381 | 0.29 |

It was found that the elasticity of product B was significantly higher than that of product A. However, the Tan delta of the two products, A and B, was relatively close: Gel B retained a significant viscous character, despite the presence of the hydroxyapatite particles (which had a high degree of elasticity and negligible viscosity).

A measurement of the normal force induced by the gel to be tested was taken by compressing the sample between the Peltier plate and the geometry for a working air gap of 1500 micrometers and a gel quantity of 1.4 g.

| Gel | Normal force (N) |
|---|---|
| A | 0.86 |
| B | 1.47 |

It was found that the elasticity of product B and its induced normal force were significantly higher than those of product A.

This higher elasticity, in combination with the strong cohesiveness of the gel according to the invention, gave the product a greater ability to create volume within tissues.

### Example 2

### Importance of the so-Called Cohesive Cross-Linked HA-Based Gel Structure

### Comparison

Gel A1 (with a so-called cohesive or monophasic structure), as described in Example 1, was dialyzed with an iso-osmolar saline solution that had a neutral pH (regenerated cellulose, separation limit: molecular weight=60 kDa) to obtain a hyaluronic acid concentration of 20 mg/ml (2%).

Calcium hydroxyapatite was then added to the gel to obtain a concentration of 200 mg/ml (20%), after which mixing was carried out using a spatula (2 minutes per 5 g of gel).

The resulting gel was then sterilized in an autoclave at 121° C. for 20 minutes (=gel B′ according to the invention).

The commercial cross-linked hyaluronic acid-based gel Restylane® Perlane® (lot 11363-1) having a so-called biphasic or non-cohesive structure, and whose hyaluronic acid concentration was 20 mg/ml (2%). was enriched with 200 mg/ml (20%) of calcium hydroxyapatite by mixing with a spatula (2 minutes per 5 g of gel).

The resulting gel was then sterilized in an autoclave at 121° C. for 20 minutes (=gel X).

Gel A1 and the Restylane® Perlane® gel were compared in the following test:

In plastic 30 ml bottles containing 5 ml of purified water, 1 ml of gel A1 was introduced into bottle 1 and 1 ml of Restylane® Perlane® gel into bottle 2. After the closing of the bottles, the 2 bottles were mixed manually for 5 seconds.

After 10 seconds, the Restylane® Perlane® gel was observed to be completely disintegrated/dispersed, in the form of a multitude of particles, in the aqueous solution. The Restylane® Perlane® gel thus possessed a so-called non-cohesive or biphasic structure (the gel quickly disperses in the aqueous solution).

Gel A1 remained in the form of a “gel ball” in the aqueous solution. It therefore did indeed have a cohesive or monophasic structure (the gel did not quickly disperse within the aqueous solution; it has strong cohesiveness, unlike the Restylane® Perlane® gel).

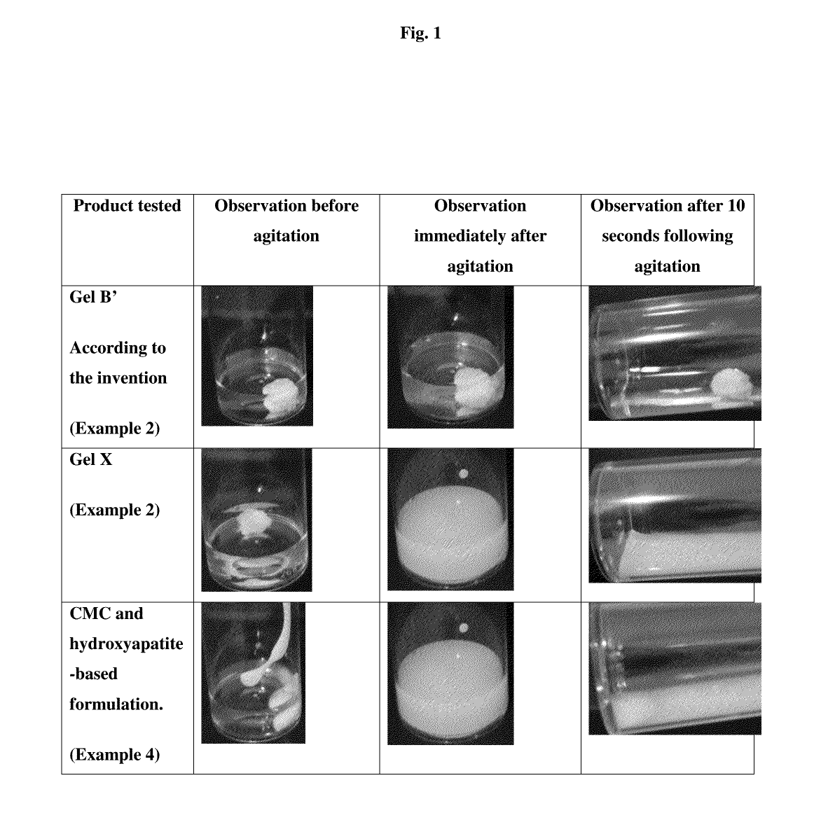

Gel B′ according to the invention and gel X were compared in the following test (see FIG. 1): In plastic 30 ml bottles containing 5 ml of purified water, 1 ml of gel B′ was introduced into bottle 1 and 1 ml of gel X into bottle 2. After the closing of the bottles, the 2 bottles were mixed manually for 5 seconds.

After 10 seconds, the Restylane® Perlane® gel was observed to be completely disintegrated/dispersed, in the form of a multitude of particles, in the aqueous solution. Gel X had a non-cohesive particle-based viscoelastic structure. It did not correspond to the characteristics of the gel according to the invention. In medical practice for use in aesthetics, it will diffuse/migrate at the injection site.

Meanwhile, gel B′ remained in the form of a “gel ball” within the aqueous solution. It therefore has a cohesive particle-based structure which, within the context of medical practice for use in cosmetic applications, prevents it from diffusing/migrating at the injection site, thereby avoiding the complications linked to the migration of hydroxyapatite particles in the tissues, while also providing better long-term product performance, because the injected gel will be able to maintain its capacity to create volume in the tissues over a long period, due to the lack of migration of the biomaterial from the treated area.

### Example 3

### Importance of the Viscoelasticity of the Gel in Accordance with the Invention

### Comparison

C is a gel prepared according to the same protocol (steps 1 & 2) described in Example 1 by introducing 200 mg of BDDE instead of 420 mg.

D is a gel prepared according to the same protocol (steps 1 & 2) described in Example 1 by introducing 290 mg of BDDE instead of 420 mg.

Gel C was characterized from a mechanical/rheological viewpoint:

The rheometer used to perform these rheological characterizations was an AR2000 (TA Instruments) with a plate geometry of 40 mm, a working air gap of 1000 micrometers, and an analysis temperature of 25° C.

A measurement of the ratio between the viscosity and the elasticity (Tan delta=G″/G′) was taken by performing a frequency sweep from 0.01 to 100 Hz.

A comparison of the parameters was performed at 1 Hz.

| Gel | Tan delta = G″/G′ (1 Hz) |
|---|---|
| C | 0.84 |
| D | 0.58 |

It was found that hydroxyapatite particles tended to settle over time (a well-known phenomenon observed by passing a sample through the centrifuge) in gel C, which phenomenon was not observed for gel D.

This sedimentation entails obtaining non-homogeneous particulate-based hydroxyapatite products. This is unsatisfactory for the act of injecting the gel through the needle (due to clogging of the needle) and also in terms of the safety and performance of the formulation in the injection zone (major risks of complications such as the creation of so-called hard areas).

As shown in Example 2, the cohesiveness of the gel according to the invention is important; however, it is also necessary for its viscoelastic nature to be suitable in order to:
Avoid the sedimentation of the hydroxyapatite particles over time in their container
Avoid having a product that will separate into 2 phases (particles of hydroxyapatite and cross-linked hyaluronic acid gel) during the injection and/or at the injection site, thus creating heterogeneities in the treated area.

Thus, the elastic nature of the gel (in relation to its viscosity) must be great enough to prevent the sedimentation of the particles.

### Example 4

### Comparison of a Gel According to the Invention Against Solutions in the Prior Art

### a) Non-Cross-Linked HA- and Hydroxyapatite-Based Formulation

As described in the literature, non-cross-linked hyaluronic acid in vivo has a half-life of less than a week.

Consequently, a solution consisting of non-cross-linked HA with hydroxyapatite is of no value, because the non-cross-linked hyaluronic acid will be absorbed very quickly and it will not prevent the migration of the hydroxyapatite particles over the long term.

### b) Aqueous Hydroxyapatite Formulation

An aqueous hydroxyapatite solution (S1) was prepared (30% phosphocalcium hydroxyapatite with a particle size of 30 to 50 micrometers in an iso-osmolar saline solution with a neutral pH).

In a plastic 30 ml bottle containing 5 ml of purified water, 1 ml of the S1 solution was added. An immediate dispersion of the hydroxyapatite particles in the bottle was observed.

Unlike the formulation according to the invention, the S1 solution was unable to hold the hydroxyapatite particles at the injection site over the long term.

### c) CMC- and Hydroxyapatite-Based Formulation (See FIG. 1).

An aqueous carboxymethyl cellulose (CMC) and hydroxyapatite (S3) formulation was prepared (30% phosphocalcium hydroxyapatite with a particle size of 30 to 50 micrometers, and 2% CMC at 250,000 Da in an iso-osmolar saline solution with a neutral pH).

In a plastic 30 ml bottle containing 5 ml of purified water, 1 ml of the S3 formulation was added. After the closing of the bottle, the bottle was manually mixed for 5 seconds.

After 10 seconds, it was observed that the S3 formulation was completely disintegrated/dispersed, in the form of a multitude of particles, in the aqueous solution.

Unlike the formulation according to the invention, the S3 solution was unable to hold the hydroxyapatite particles at the injection site over the long term.

1. A sterile aqueous injectable formulation, sterilized by moist heat, used for therapeutic purposes, in the form of a cohesive particle-based viscoelastic gel containing:
i) cross-linked hyaluronic acid or one of its salts at a concentration between 1% and 4% (mass/volume); wherein the cross-linking that is performed makes it possible to obtain a gel having a base of cross-linked hyaluronic acid having a cohesive structure, and
ii) hydroxyapatite at a concentration between 10% and 70% (mass/volume), wherein the hydroxyapatite is in the form of particles having an average size in a range of 500 nm to 80 μm;
and further wherein the sterile injectable aqueous formulation has viscoelastic properties such that Tan δ at a frequency of 1 Hz is less than or equal to 0.60.

2. The sterile injectable aqueous formulation of claim 1, wherein the molecular weight of the hyaluronic acid, or of one of its salts, is between 2.5×10⁵ Da and 4×10⁶ Da.

3. The sterile injectable aqueous formulation of claim 1, wherein the concentration of the cross-linked hyaluronic acid or of one of its salts is between 1% and 3% (mass/volume).

4. The sterile injectable aqueous formulation of claim 1, wherein the concentration of the hydroxyapatite is between 20% and 60% (mass/volume).

5. (canceled)

6. The sterile injectable aqueous formulation of claim 1, wherein the formulation further comprises one or more ceramic materials.

7. The sterile injectable aqueous formulation of claim 6, wherein the ceramic material is tricalcium phosphate.

8. The sterile injectable aqueous formulation of claim 1, wherein the formulation further comprises one or more anesthetics.

9. The sterile injectable aqueous formulation of claim 8, wherein one or more anesthetics are selected from the group consisting of lidocaine alone or in combination with adrenaline; procaine; etidocaine alone or in combination with adrenaline; articaine alone or in combination with epinephrine; mepivacaine; pramocaine; and quinisocaine; or one or more salts thereof.

10. The sterile injectable aqueous formulation of claim 9, wherein the anesthetic is lidocaine hydrochloride.

11. The sterile injectable aqueous formulation of claim 1, wherein the formulation further comprises one or more antioxidants.

12. The sterile injectable aqueous formulation of claim 11, wherein one or more antioxidants are selected from the polyol family.

13. The sterile injectable aqueous formulation of claim 12, wherein the polyol is selected from the group consisting of sorbitol, glycerol, mannitol, and propylene glycol.

14. The sterile injectable aqueous formulation of claim 1, wherein the formulation further comprises one or more growth factors.

15. (canceled)

16. (canceled)

17. A kit comprising the sterile injectable aqueous formulation of claim 1.

18. The kit of claim 17, in the form of a syringe, ampoule, or bottle.

19. A method for making a sterile aqueous injectable formulation, comprising the following steps:
a) preparing a first mixture containing at least 1% to 4% by weight of cross-linked hyaluronic acid or a salt thereof, by the formation of covalent bonds between the chains of the biopolymer, with the aid of bi- or polyfunctional molecules, wherein the cross-linking that is performed makes it possible to obtain a gel having a base of cross-linked hyaluronic acid possessing a monophasic or cohesive structure,
b) purifying the first mixture,
c) adding hydroxyapatite at a concentration between 10% to 70% (mass/volume), by homogeneously dispersing the hydroxyapatite in the cross-linked hyaluronic acid-based gel,
d) placing the gel in a ready-to-use form, and
e) sterilizing the product with moist heat.

20. The sterile injectable aqueous formulation of claim 14, wherein the one or more growth factors is selected from the group consisting of “Bone morphogenetic proteins” (BMPs) and “transforming growth factors β” (TGF-βs) and a combination thereof.

21. The sterile injectable aqueous formulation of claim 1, wherein the viscoelastic properties are determined by performing a frequency sweep from 0.01 to 100 Hz using a rheometer with a plate geometry of 40 mm, a working air gap of 1000 micrometers, and an analysis temperature of 25° C.
